(19) United States
(12) Plant Patent Application Publication
(10) Pub. No.: US 2026/0157249 P1
(43) Pub. Date: Jun. 4, 2026

(54) Aechmea plant named 'A4006'

(50) ;

(71) Applicant: Chester Skotak, JR., Alajuela (CR)

(72) Inventor: Chester Skotak, JR., Alajuela (CR)

(21) Appl. No.: 19/401,699

(22) Filed: Nov. 26, 2025

(30) Foreign Application Priority Data
Nov. 29, 2024 (QZ) 2024/2860

(51) Int. Cl. A01H 6/22; A01H 5/02; A01H 5/12

(52) U.S. Cl. CPC A01H 6/22; A01H 5/02; A01H 5/12
USPC PLT/370

(57) ABSTRACT

A new and distinct Aechmea plant named ‘A4006’ particularly distinguished by leaves entirely lacking marginal spines; a very compact rosette plant habit, upright inflorescence, positioned well above the foliage; a uniform plant form and high stability through asexual propagation; distinctive bright pink bracts with violet-blue flowers.

Genus and species: Aechmea fasciata.

Variety denomination: ‘A4006’.

### STATEMENT REGARDING PRIOR DISCLOSURES BY THE INVENTOR

This application claims priority to CPVO application no. 2024/2860 filed on Nov. 29, 2024, which is herein incorporated by reference. The Applicant asserts that no publications or advertisements relating to sales, offers for sale, or public distribution occurred more than one year prior to the effective filing date of this application. Any information about the claimed plant would have been obtained from a direct or indirect disclosure from the Inventor. The Applicant claims a prior art exemption under 35 U.S.C. 102(b)(1) for disclosures and/or sales that fall within a one-year grace period prior to the effective filing date.

### BACKGROUND OF THE NEW PLANT

The present invention comprises a new and distinct variety of Aechmea plant, botanically known as Aechmea fasciata, and hereinafter referred to by the variety name ‘A4006’.

Aechmea comprises a genus of more than 168 species of evergeen perennials suitable for cultivation in the home or in the greenhouse. Aechmea may be terrestrial or epiphytic. For the most part, the species vary in diameter from 12 to 18 inches to 3 or 4 feet and have rosettes of spiny-edged leaves.

Flowers and bracts of Aechmea frequently have brilliant colors and may last for several months. The range of colors for Aechmea is generally from the yellow through orange but may also include pink, orange, red and red-purple. Tubular, three-petalled flowers may also appear but are usually short-lived.

Aechmea may be grown as pot plants for greenhouse or home use. Typically, the plants are shaded from direct sunlight; and the central, vaselike part of the leaf rosette is normally filled with water.

Aechmea is native to tropical America. Leaves of Aechmea are usually formed from basal rosettes which are entire and in several vertical ranks. Aechmea has terminal spikes or panicles which are often bracted with petals united in a tube longer than the calyx.

Asexual propagation of Aechmea is frequently done through meristematic tissue culture. Propagation can also be from offshoots which can be detached from the mother plant and grown in an appropriate soil or bark mixture. Methods for cultivating and crossing of Aechmea are well known.

The new Aechmea variety ‘A4006’ is the product of a controlled cross pollination conducted by the inventor in Alajuela, Costa Rica. The objective of the breeding program was to develop compact, spineless, and uniform Aechmea fasciata plants suitable for pot production with upright inflorescence and improved ornamental presentation. The female or seed parent was an unnamed and unpatented Aechmea fasciata breeding line. The male or pollen parent was an unnamed and unpatented Aechmea fasciata breeding line. ‘A4006’ was selected by the inventor in 2021 in Alajuela, Costa Rica as a single flowering plant from within the progeny of the stated cross.

The new Aechmea cultivar “A4006’ was first reproduced asexually by meristematic tissue culture at a commercial laboratory in Evergem, Belgium. Asexual propagation by meristematic tissue culture of the new variety has shown that the unique features of the new variety are stable and reproduced true-to-type to date through successive generations.

### SUMMARY OF THE INVENTION

The new Aechmea cultivar ‘A4006’ has not been observed under all possible environmental conditions. The phenotype may vary somewhat with variations in environment such as temperature, day length, light intensity, water status, fertilizer rate and type, without, however, any variance in genotype.

The following are the most outstanding and distinguishing characteristics of this new Aechmea cultivar. The combination of these characteristics distinguishes ‘A4006’ as a new and distinct variety of Aechmea.
1. Aechmea ‘A4006’ exhibits leaves entirely lacking in marginal spines.
2. Aechmea ‘A4006’ exhibits a very compact rosette plant habit.
3. Aechmea ‘A4006’ exhibits a fully upright inflorescence, positioned well above the foliage.
4. Aechmea ‘A4006’ exhibits a uniform plant form and high stability through asexual propagation.
5. Aechmea ‘A4006’ exhibits distinctive bright pink bracts with violet-blue flowers.

### DESCRIPTION OF THE PHOTOGRAPHS

This new Aechmea cultivar is illustrated by the accompanying-colored photographs which show the overall appearance and distinct characteristics of the plant. The colors shown are as true as can be reasonably obtained by conventional photographic procedures. The photographs are of 24 to 28-month-old plants grown in 14-centimeter circular containers in a conventional glass greenhouse in Evergem, Belgium. Colors in the photographs may differ slightly from the color values cited in the botanical description which accurately describes the colors of the new variety.

FIG. 1. shows the overall plant form, foliage, and inflorescence of ‘A4006’.

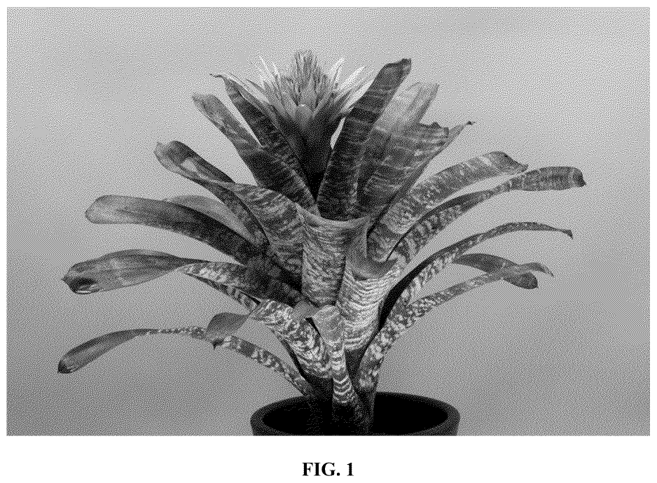

FIG. 2. shows a close-up of typical foliage observed on plants of ‘A4006’.

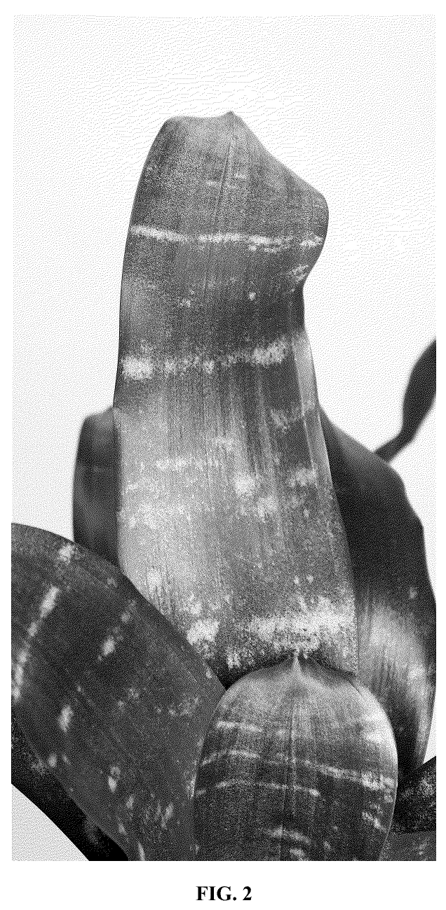

FIG. 3. shows an overhead view of a typical inflorescence observed on plants of ‘A4006’.

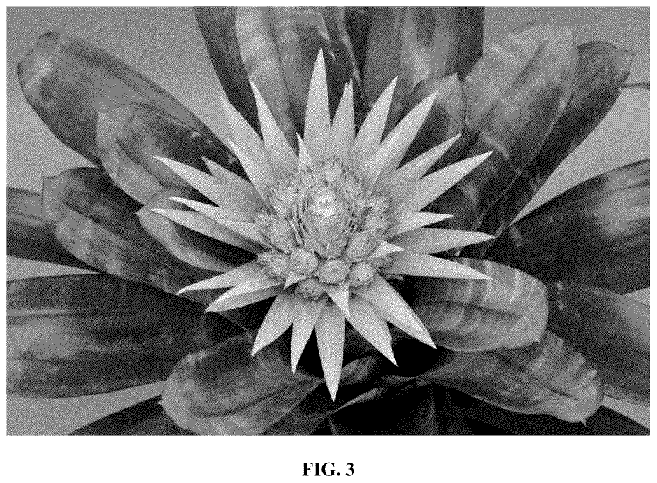

FIG. 4. shows a side view of a typical inflorescence observed on plants of ‘A4006’.

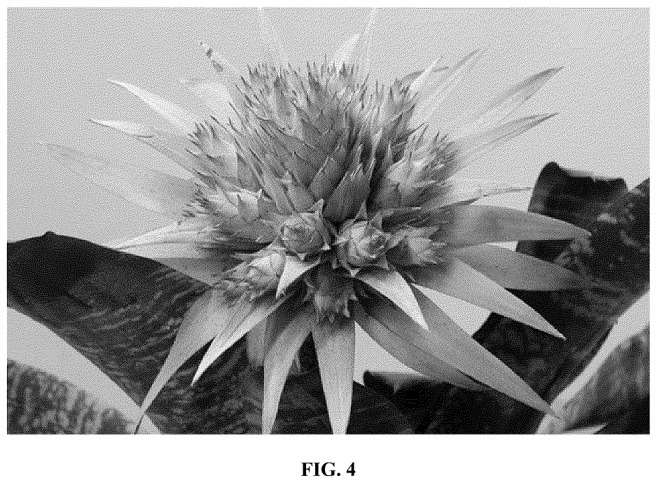

### DESCRIPTION OF THE NEW VARIETY

In the following description, color references are made to The Royal Horticultural Society Colour Chart, Sixth Edition, except where general color terms of ordinary dictionary significance are used.

The following observations and measurements describe plants grown in a commercial greenhouse located in Evergem, Belgium under normal light levels with no supplemental lighting. Average daily temperatures ranged between 21° C. and 23° C. and average nightly temperatures ranged between 19° C. and 21° C. Measurements and numerical values represent averages of typical plant types.

### DETAILED BOTANICAL DESCRIPTION

Classification:
   Family.—Bromeliaceae.
   Botanical.—Aechmea fasciata.
   Denomination.—‘A4006’.
General Description:
   Plant type.—Tropical perennial suitable as an indoor potted plant.
   Growth habit.—Highly symmetrical, compact and dense basal rosette; inflorescence grows from the center of the basal rosette.
   Overall plant shape.—Flattened obovate with inflorescence carried well above above the foliar plane.
   Height from soil level to top of foliar plane.—Average 27.0 cm.
   Plant diameter or area of spread.—Average 40.4 cm.
   Growth rate.—Moderate.
   Plant vigor.—Moderate.
   Branching habit.—No lateral branching; leaves in a basal rosette, inflorescence grows from the center of the basal rosette.
   Propagation type.—Meristematic tissue culture.
Root description:
   General.—Slightly fleshy and very slightly fibrous.
   Length.—Average 5.4 cm.
   Width.—Average 0.1 cm.
   Color.—165B (greyed-orange).
Foliage description:
   Arrangement.—Rosulate.
   Compound or Single.—Single.
   Quantity of leaves per rosette.—Average 21.
   Lamina.—Shape: Oblong. Length: Average 21.3 cm. Width: Average 4.5 cm Aspect: Slightly concave, curled downward on average. Apex shape: Truncate to broadly retuse with a mucronulate tip. Base: Sheathing. Sheath length: Average 6.1 cm. Sheath width Average 5.1 cm. Sheath color: 148C (yellow-green) and lighter towards the edge, 144B (yellow-green). Margin: Entire, spinless. Undulation: Coarsely undulate and slightly twisted axially. Lobes: Unlobed. Texture (both upper and lower surfaces): Smooth and glabrous. Rugosity: Non-rugose. Pubescence: No pubescence present. Luster: Upper surface: Glossy. Lower surface: Slightly glossy. Color: Juvenile foliage: Upper surface: 147A (yellow-green) with edges colored NN137B (green) and heavily blotched lighter than NN155D (white) with a silvery haze loosely arranged in radial lines across the lamina. Lower surface: NN137A (green) with lighter edges colored 137B (green) and heavily blotched lighter than NN155D (white) with a silvery haze loosely arranged in radial lines across the lamina. Mature foliage: Upper surface: NN137A (green) and blotched NN155D (white) with a silvery haze loosely arranged in radial lines across the lamina; significantly less white blotching on the upper half of the lamina on mature leaves. Lower surface: 147A (yellow-green) and heavily blotched lighter than NN155D (white) with a silvery haze loosely arranged in radial lines across the lamina. Venation: Pattern: Parallel. Color (mature foliage): Upper surface: Upper surface venation is colored the same as the upper surface of the lamina. Lower surface: Lower surface venation is colored the same as the lower surface of the lamina.
Inflorescence description:
   Type.—Panicle.
   Length.—39 cm.
   Length of flowering portion.—Average 101 mm.
   Width of flowering portion.—Average 150 mm.
   Number of flowers per bract.—Average 22.
   Peduncle shape in cross section.—Round.
   Largest bract.—Length: Average 101 mm. Width: 25 mm. Main color (upper surface): 84B (light purple). Shape of tip: Acuminate.
   Floral bract.—Length: Average 100 mm. Width: Average 20 mm. Main color (upper surface): 84B (light purple). Main color of top: 84B (light purple). Shape: Ovate. Profile in cross section: Medium concave. Shape of top: Acute.
   Prophyll.—Length: Average 15 mm. Width: Average 9 mm. Main color: 84B (light purple).
   Flower merosity.—3.
   Calyx.—Length: Average 14 mm. Width: Average 6 mm. Color: 84C (light purple).
   Petal.—Length: Average 8 mm. Width: Average 15 mm. Color: 90C (violet), turning more red with age. Shape of tip: Rounded.
   Stamen.—Length: Average 15 mm. Color: White.
   Anther color.—White.
   Style.—Length: Average 20 mm. Color: White.
   Stigma color.—White.
   Ovary color.—White.
Fruit and seed set: None observed to date.
Temperature tolerances: USDA Zones 10 to 13; at least tolerant of temperatures to 40 degrees Celsius.
Rain tolerance: High.
Wind tolerance: High.
Disease and pest tolerance: No claims are made to any superior disease or insect resistance or tolerance.

### Comparison With the Parent Plants

The new Aechmea cultivar ‘A4006’ can be distinguished from its female or seed parent in the following characteristics described in Table 1 below.

TABLE 1

| Characteristic | ‘A4006’ | Female parent |
|---|---|---|
| Overall plant size | Smaller, compact rosette | Larger, open rosette |
| Leaf margin | Entirely spineless | Finely spined |
| Leaf color | Green with a stronger | Green-grey with |
| | silver hue | faint silver |
| | | banding |
| Leaf shape | Broad, slightly recurved | Narrow, arching |
| Inflorescence position | Fully upright above foliage | Partly within rosette |
| Floral bract color | Bright pink | Pale pink |
| Growth habit | Dense and upright | Open and spreading |

The new Aechmea cultivar ‘A4006’ can be distinguished from its male or pollen parent in the following characteristics described in Table 2 below.

TABLE 2

| Characteristic | ‘A4006’ | Male Parent |
|---|---|---|
| Plant size | Small and compact | Medium to large |
| Leaf margin | Spineless | Short spines |
| Leaf variegation | Subtle silver hue | Moderate |
| Inflorescence position | Fully upright | Slightly inclined |
| Bract color | Deep pink | Light pink |
| Flower arrangement | Dense, centered cluster | Looser |
| Overall presentation | Highly symmetrical | Less symmetrical |

What is claimed is:

1. A new and distinct cultivar of Aechmea plant named ‘A4006’, substantially as illustrated and described herein.
